(19) United States
(12) Patent Application Publication
(10) Pub. No.: US 2008/0255417 A1
(43) Pub. Date: Oct. 16, 2008

(54) METHOD FOR REDUCING THE FRICTION OF A MEDICO-TECHNICAL RUBBER TUBE

(75) Inventor: Konstantin Bob, Weinheim (DE)

Correspondence Address: STANDLEY LAW GROUP LLP, 495 METRO PLACE SOUTH, SUITE 210, DUBLIN, OH, 43017, US

(73) Assignee: INVENDO MEDICAL GMBH, Weinheim (DE)

(21) Appl. No.: 12/099,596

(22) Filed: Apr. 8, 2008

(30) Foreign Application Priority Data
Apr. 10, 2007 (DE) 102007000214.0

(51) Int. Cl. A61B 1/01; B29C 71/00; B29C 71/02

(52) U.S. Cl. 600/114; 264/233; 264/130

(57) ABSTRACT

There are described a method for reducing the friction of a medico-technical rubber tube, according to which first a tube is produced of rubber and is then after-treated by annealing and/or washing, as well as an endoscope.

### CROSS-REFERENCE TO RELATED APPLICATIONS

This application claims a right of priority under 35 USC §119 from German patent application 10 2007 000 214.0, filed 10 Apr. 2007, the content of which is incorporated by reference as if fully recited herein.

### TECHNICAL FIELD

The present invention relates to a method for reducing the friction of a medico-technical rubber tube as well as to an endoscope for which a medico-technical rubber tube is used.

### BACKGROUND OF THE ART

Devices for introducing a medical endoscope into a body canal are described, for instance, in commonly-owned U.S. Pat. No. 5,259,364, the content of which is incorporated by reference as if fully recited herein. The devices described in this document permit that an endoscope is no longer pushed into the body to be examined, but moves into the body all by itself. For this purpose, the endoscope is equipped with an inherent drive which allows easier and quicker insertion.

As such an inherent drive, also a so-called everting tube can be used, for instance, with the endoscope shaft being inserted therein. Upon propulsion of the endoscope different relative movements occur. On the one hand, a relative movement occurs between the endoscope shaft and the everting tube which are in sliding contact with each other. On the other hand, there is also a relative movement between an inside portion and an outside portion of the unwinding everting tube.

In the current development of such devices, there is the endeavor to manufacture tubes adapted to achieve a reduced friction between e.g. an inserted everting tube and an endoscope shaft, between the everting tube and a cover of the endoscope shaft as well as between individual portions of an everting tube.

If, for instance, silicone tubes are used, stickiness of the surface, the so-called “tag”, can readily be observed due to the formation of decomposition products in manufacture. This “tag” is opposed to the reduced friction aimed at, so that, so far, only hesitant use has been made of silicone tubes in medico-technical devices requiring low frictions, such as in endoscopic applications, in particular.

### SUMMARY OF THE INVENTION

The present invention has been made in view of the endeavors mentioned above.

It is an object of the invention to provide a method for manufacturing a medico-technical rubber tube whose friction and “tag” are reduced.

The method should be as simple as possible, since such a rubber tube may be a disposable, so that the manufacture thereof should be favorably priced.

In addition, the method should be adaptable to different tube materials in the easiest possible manner.

It is further desirable that the method supplies a tube that can be easily made to adhere to, or rather be coated with further materials such as supplementing lubricants.

It is a further object of the invention to provide a suitable rubber tube as such, whose sliding properties are improved as described above, which exhibits less “tag”, is favorably priced, and can be excellently combined with various further lubricants.

It is still another object of the present invention to provide an advantageous application of such a tube having reduced friction.

For achieving the above-mentioned object, the present invention provides a method for reducing the surface friction coefficient (μ) of a medico-technical rubber tube, comprising the steps of:
a. manufacturing a tube of rubber material, and
b. finish-treating of the tube by annealing and/or washing, wherein the finish-treatment effects a change of the material condition at least at the surface of the tube and
c. optionally applying a lubricant to the tube surface as an additional coating process.

The rubber material is preferably selected from silicone material, PTFE material, ePTFE material and thermoplastics, preferably thermoplastic polyurethane, into which preferably an oil is worked.

In a further preferred embodiment, the silicone material is getting addition cross-linked with a platinum compound in manufacture.

In a further preferred embodiment, the silicone material is cross-linked peroxidically in manufacture.

Preferably, in manufacture, the rubber material is extruded to form a tube.

When this is done, it is particularly preferred that, either in the extrusion process itself or after the extrusion process, knobs are generated in a separate step on at least one tube surface.

The knob generated in the extrusion process is e.g. a continuous spirally circumferential projection.

In the subsequent treatment, annealing is preferably effected at 120 to 180° C.

In the subsequent treatment, annealing is preferably effected for 30 to 180 minutes.

In addition, in the subsequent treatment, washing preferably takes place at 30 to 90° C.

Moreover, in the subsequent treatment, washing preferably takes place for 30 to 60 minutes.

Washing is preferably effected by means of an aqueous, alkaline washing liquid.

In a further preferred embodiment, the tube is coated with a lubricant following the subsequent treatment.

When this is done, the lubricant is preferably selected from agar-agar, vegetable oil, a fat-wax mixture, (e)PTFE lubricating varnish, (e)PTFE powder, graphite, talcum, further silicone coatings different from the silicone material of the tube, silicone oil, graphite and/or Teflon powder dispersed in lubricating oil, as well as (e)PTFE and/or glass beads dispersed in lubricating oil, or from a combination of two or more of these.

In a preferred embodiment, the surface of the tube is set with (e)PTFE beads and/or glass beads subsequent to the after-treatment (but prior to the possible method step of coating with a lubricant).

In particular, the tube of the methods described is a shaft cover for an endoscope or an everting tube for an endoscope. According to the invention, there is further provided a medico-technical rubber tube obtainable by the method according to the invention.

The present invention moreover provides an endoscope having an everting tube drive, wherein the endoscope (or rather the endoscope shaft or an endoscope shaft cover put thereover) and/or the everting tube consist/consists of a rubber material, and knob-shaped projections are formed on at least one of the surfaces of the everting tube and/or of the surface of the endoscope.

The everting tube uncoils on the endoscope shaft cover.

Preferably, the rubber material is selected from silicone material, PTFE material, ePTFE material and thermoplastics, preferably thermoplastic polyurethane, into which preferably an oil is worked.

It is further preferred that there is a wax-containing fat or an oil between and/or in the knob-shaped projections. It is preferred that the knob-shaped projections are formed as perforated lubricant reservoirs.

In a preferred embodiment, the knob-shaped projections are formed by beads made of (e)PTFE or glass, which adhere to the respective surface.

The preferred diameter of the beads lies within the range of ≧0.1 to ≦0.2 mm.

Supplementary objects, advantages and preferred embodiments are to be gathered from the ensuing detailed description of the invention.

### DETAILED DESCRIPTION OF THE INVENTION

The method according to the invention for reducing the surface friction coefficient (μ) of a medico-technical rubber tube first requires the step of manufacturing a tube. When doing so, the rubber material is preferably selected from silicone material, PTFE material, ePTFE material and thermoplastics, preferably thermoplastic polyurethane, into which preferably an oil is worked.

The silicone material preferably used is, just as the other above-mentioned types of rubber, basically well suited for medico-technical applications by its inert properties; however, in the polymerization itself or later by environmental impact, different decomposition products such as alcohols, acids, derivatives thereof, polymer fragments, etc. are produced, also in dependence on the groups bound to the silicon atoms of the starting materials. Such decomposition products often lead to an increased adhesion and, consequently, to an increased friction of the tubes produced. Also, in the manufacturing process, undesired oily residues may be separated, these may lead to a reduced adhesion between tube and coating, or a complete lack thereof, in a subsequent coating, e.g. with a lubricant.

A first measure for reducing the decomposition product quantity consists in subjecting the silicone material in the manufacturing process to an addition cross-linking, preferably an addition cross-linking with platinum compounds being used. For this, particularly Pt(0) complexes are suited, they may e.g. comprise vinyl siloxanes as ligands.

Due to the clearly reduced generation of decomposition products by the addition cross-linking, friction is advantageously reduced. A tube manufactured from such an addition cross-linked silicone material is especially well suited as everting tube used for forward propelling a device inserted therein, e.g. the shaft (with or without cover) of an endoscope.

It is a further advantageous measure for reducing the decomposition product quantity to peroxidically cross-link the silicone material in the manufacturing process. In a peroxidic cross-linking, e.g. alkyl or aryl peroxides such as dicumyl peroxide, 1,4-bis(tert-butylperoxy)-1,4-dimethyl hexane, 2,4-dichlorobenzoyl peroxide and 4-methyl benzoyl peroxide can be used. Although a peroxidic cross-linking supplies slightly larger amounts of a decomposition product than an addition cross-linking, the part thereof is still greatly improved compared to a non-cross-linked silicone material. Moreover, the peroxidic cross-linking is extremely favorably priced.

For this reason, the peroxidic cross-linking is preferably used in case of the tube to be manufactured being a disposable, e.g. a cover to be arranged over the shaft of an endoscope (shaft cover).

In particular when the medico-technical rubber tube of the present invention is used as an everting tube, silicone material is particularly well suited as basic substance for this tube, because it is comparably favorable in price and can be easily worked upon such that the reduction of μ aimed at can also be achieved specifically in the everting areas.

If the medico-technical rubber tube of the present invention is used as a shaft cover, also a thermoplastic rubber is very well suited, preferably a thermoplastic polyurethane. Hereby, a good connection to the actual shaft with a simultaneous friction reduction towards the outside can be achieved.

The formation of the rubber material in tube form can be effected by methods of rubber processing which are known per se. According to the invention, an extrusion process is preferred, since in this way any desired special cross-sectional shapes and/or profiles can be conferred upon the tube. Advantageously, in such an extrusion process, knob-shaped projections are generated on at least one tube surface. “Tube surface” means the outer and the inner (shell) surfaces of the tube in this case.

In the extrusion process, a continuous, spirally circumferential knob (projection) on the tube surface can be formed particularly well.

It is further possible to generate the knobs in a separate step following the extrusion, e.g. by cutting, punching, embossing, laser engraving, or the like. In these processes, the material remaining between the knobs is normally removed. The knobs may, however, also be generated in the separate step from a smooth surface, e.g. by foaming special surface areas, for instance by local, e.g. point-shaped, heating of the surface (e.g. by a laser).

Such knob-shaped projections (or knobs), e.g. a spiral-shaped knob, on the surface serve as spacers on the tube surface between which a lubricant can be filled in.

It is also possible that the lubricant (wax-containing fat, oil such as silicone oil, vegetable fat or oil) is alternatively or supplementarily available in the knobs themselves, which then serve as lubricant reservoir. The lubricant may get out of these lubricant reservoirs, e.g. upon the application of pressure. The discharge of the lubricant may further be simplified by the lubricant reservoirs being perforated. Perforation may be effected simultaneously with the generation of knobs, or in a further procedural step.

In both cases, i.e. lubricant between and in the knobs, the slidability of the surface is again improved considerably. The lubricant can be applied onto the knob surface produced in this way, or after the knob surface has been coated with a lubricating varnish, e.g. with an (e)PTFE lubricating varnish, or has been plasma-treated/plasma-coated. The lubricant is applied in dependence on its chemical and physical properties before or after the subsequent treatment of the method according to the invention, which shall be described later. Suitable lubricants shall be described later, as well.

In the method according to the invention, the manufacture of the tube from rubber material is followed by an after-treatment of the tube by annealing and/or washing. Both after-treatments are adapted clearly to reduce the amount of decomposition products and oily residues still existing on the surface, which thus leads to an improvement of the sliding properties and a better adhesion to a coating applied later, respectively. The best results are achieved if both after-treatments are carried out in a random order.

If required, the rubber can additionally be vulcanized with typical vulcanization means, e.g. sulfur, sulfur-containing compounds, peroxides, and zinc compounds such as zinc white, being used.

If annealing takes place, this is preferably effected at 120 to 180° C. At lower temperatures, the decomposition products/oily residues are sometimes only removed insufficiently, whereas higher temperatures do not provide any further improvements and can, in some cases, damage the rubber structure.

Furthermore, it is preferred in the case of annealing, to do so for 30 to 180 minutes. At shorter annealing times, there is the possibility that the decomposition products/oily residues are only removed insufficiently, whereas longer annealing times do mostly not result in any further reduction of the “tag”, and are then uneconomical.

The temperatures in annealing and the annealing times are preferably selected in dependence on the cross-linking method used. In particular, for addition crosslinked silicone materials, this serves to improve the compressive rigidity of the silicone tube obtained, which is of advantage, particularly when using the same as everting tube. For this, the preferred annealing temperature ranges from 120 to 140° C., as annealing time the range from 30 to 60 minutes is to be preferred. The best results are obtained when annealing time and annealing temperature are simultaneously selected from the indicated ranges. A significant improvement of the compressive rigidity of the tube is e.g. obtained for the preferred combination of 140° C./30 minutes.

On the other hand, the described advantages of the annealing process, e.g. with peroxidically crosslinked silicone materials, are reliably achieved if slightly higher temperatures of preferably 160 to 180° C. are applied. For the same reason, annealing is preferably effected for 150 to 180 minutes. The best results are, in turn, obtained if both preferred ranges are simultaneously kept, e.g. by the combination of 180° C./180 minutes.

In case of washing, washing is preferably carried out at 30 to 90° C. At lower temperatures, the decomposition products/oily residues are sometimes not removed adequately, whereas higher temperatures do often not yield any better effect, and only generate higher cost.

Moreover, if washing takes place, this is preferably done for 30 to 60 minutes. If the washing times are shorter, the removal of the decomposition products/oily residues is easily insufficient, if the washing times are longer, they do mostly not have any further effect.

Washing can simply be done in a customary washing machine. As washing liquid, an aqueous washing liquid is preferably used, preferably a slightly alkaline washing liquid (pH>7 and ≦9). Very good results can be achieved already with customary washing agents for clothing. In a test carried out concretely, a silicone tube manufactured according to the invention was put into a washing machine and washed with water, with 30 g NaOH being added. The washing operation was carried out at 60° C. for 45 minutes. Upon completion of the washing operation, there was a waiting time of 15 minutes, afterwards the washed tube was washed with distilled water until neutrality.

The tube produced by the above-described method is very well suited as such for medico-technical applications, particularly in endoscopy. In order to achieve a further improvement of the sliding properties, it is often an advantage to apply a further lubricant onto the tube surfaces exposed to friction. Especially compatible lubricants which adhere well to the rubber are agar-agar, (e)PTFE lubricating varnish, talcum, graphite, further silicone coatings different from the silicone material of the tube (e.g. applied by plasma coating), silicone oil, graphite powder and/or Teflon powder dispersed in lubricating oil, as well as (e)PTFE and/or glass beads dispersed in lubricating oil, or a combination of two or more of the cited lubricants.

Instead of an (e)PFTFE lubricating varnish, also an (e)PTFE powder can be used (nano particles). This powder is preferably applied by plasma coating. To improve adhesion on the tube, the latter can be etched prior to the application of the powder, preferably by means of plasma etching.

The good adhesion of the additional lubricant on the tube is surprisingly achieved by a pre-treatment (annealing and/or washing), i.e. not only the “tag” of the tube is reduced, but, in addition, its compatibility over other lubricants is improved. For instance, regarding the adhesion of agar-agar, it has been found that this can be improved over a tube that is not after-treated (=100%) after annealing and washing to up to 150%.

The endoscope described in the present invention comprises an everting tube drive, wherein the endoscope (i.e. the endoscope shaft or an endoscope shaft cover put thereon) and/or the everting tube consist/consists of a rubber material which is preferably selected from the above-cited ones. To improve the sliding properties, knob-shaped projections are formed on at least one of the surfaces of the everting tube and/or on the surface of the endoscope (shaft or shaft cover). It has become apparent that such a structure leads to a clear reduction of μ.

Special embodiments of the knob-shaped projections are the knobs mentioned above, which can e.g. be produced directly in an extrusion process or in a subsequent, separate method step, and beads made of (e)PTFE or glass, which adhere to the surface, respectively. That is, the beads slide on the opposite sliding surface, but do basically not move on the tube itself.

Advantageous bead diameters range from ≧0.1 to ≦0.2 mm. Managing the application is sufficiently easy in this range, and additionally the optimum lubricating effect results.

The use of such beads causes a further reduction of the friction, resulting in clearly improved sliding properties in a friction of tube on tube as well as in a friction of tube on shaft. One reason for this improvement is a clearly reduced abutting surface, i.e. only the tangential area of the beads.

Moreover, the beads serve as spacers between which a further lubricant can be filled in. In this respect, the effect of the adhered beads corresponds to the above-mentioned knobs, i.e. these two embodiments may be regarded as alternatives of the knob-shaped projections, having the same effect and following the same concept.

Lubricants in both embodiments may be the above-indicated lubricants as well as, in particular, non-hydrated or partly to completely hydrated vegetable oils, such as rapeseed (canola) oil and sunflower oil.

Also a wax-containing fat or fat mixture is particularly well suited, such as an oil or oil mixture, preferably a mixture of vegetable oil and wax.

It has become apparent that the use of a 50:50 mixture from completely hydrated rapeseed oil and highly oleic-acid-containing sunflower oil (so-called HO sunflower oil), said mixture having a solidification point within the range of preferably 25 to 50° C., more preferably 30 to 40° C., and in particular preferably 35 to 39° C. as well as a clear melting point of preferably 50 to 60 and more preferably 54 to 56° C., has very good sliding properties, and can be used in endoscopic applications while showing a good compatibility.

In order to further improve the adhesion of such a mixture to the rubber tube (either the rubber tube as such or the rubber tube having knob-shaped projections), preferably a certain amount of wax has been added to the mixture or is already contained therein, for instance in non-winterized oil (non-fractioned oil).

An above-described fat, fat mixture, oil or oil mixture can also be entered into the rubber material itself, preferably into the rubber material made of thermoplastic rubber, particularly that made of thermoplastic polyurethane.

As has been mentioned above, the method according to the invention is particularly directed to the manufacture of a rubber tube forming a shaft cover for an endoscope or an everting tube for an endoscope. Accordingly, the present invention also covers a medico-technical rubber tube obtainable by the method according to the invention.

In tests in which test persons were subjected to an actual endoscopy, the two below-described kinds of silicone tubes have proved to be particularly suitable, they form specific examples of embodiments of the present invention.

On the one hand, the endoscopy could be carried out on the patient in a much simpler way compared to a conventional device (i.e. it was quicker, simpler in use, and considerably more agreeable to the patient examined) by the use of an everting tube as means of propulsion, for which a medico-technical silicone tube was used, said tube comprising knobs on at least one of its surfaces, the external surface in the specific case.

It has been found in a supplementary test that a silicone tube with a wax-containing fat between the knobs (the above-mentioned 50:50 mixture of rapeseed oil and sunflower oil having a solidification point of approx. 37° C. and a clear melting point of approx. 55° C.) has better sliding properties than one without this fat.

Similarly, in a second study, a conventional endoscope was compared with an endoscope for which an everting tube composed of a rubber tube (here: made of silicone) according to the invention with beads on its outer surface was used. The endoscope operated with the tube according to the invention again brought about the advantages of the examination being quicker, use by the examining person being safer, as well as the patient's feeling being better. These advantages could be achieved for beads made of PTFE, ePTFE and glass.

Tests made with different bead diameters provided the best sliding properties for bead diameters ranging from 0.1 to 0.2 mm.

The use of the above-described rubber tubes as shaft covers for an endoscope or as everting tubes for an endoscope is especially advantageous due to the excellent sliding properties, the compatibility with different lubricants, the low cost involved, and the ease of manufacture, and such a use is also covered by the invention.

What is claimed is:

1. A method for reducing the surface friction coefficient (μ) of a medico-technical rubber tube, comprising the steps of:
manufacturing a tube of rubber material, and
finish-treating the tube further by at least one of: annealing and washing.

2. The method according to claim 1, wherein:
the rubber material is selected the group consisting of: silicone, PTFE, (e)PTFE and thermoplastics, preferably thermoplastic polyurethane, into which preferably an oil is worked.

3. The method according to claim 2, wherein:
the manufacturing step comprises addition cross-linking of the silicone material with a platinum compound.

4. The method according to claim 2, wherein:
the manufacturing step comprises peroxidically cross-linking the silicone material.

5. The method according to claim 1, wherein:
the manufacturing step is achieved by extruding the rubber material.

6. The method according to claim 5, wherein:
the extruding step comprises the step of generating knobs on at least one tube surface.

7. The method according to claim 5, wherein:
the extruding step is followed by a step of generating knobs on at least one tube surface.

8. The method according to claim 1, wherein:
the annealing step takes place at 120 to 180° C.

9. The method according to claim 1, wherein:
the annealing step takes place for 30 to 180 minutes.

10. The method according to claim 1, wherein:
the washing step takes place at 30 to 90° C.

11. The method according to claim 1, wherein:
the washing step takes place for 30 to 60 minutes.

12. The method according to claim 1, wherein:
the washing step is effected in an aqueous, alkaline washing liquid.

13. The method according to claim 1, further comprising the step of:
coating the treated tube with a lubricant, after the finish-treatment step.

14. The method according to claim 13, wherein:
the lubricating step is achieved with a lubricant selected the group consisting of: agar-agar, vegetable oil, a fat-wax mixture, (e)PTFE lubricating varnish, (e)PTFE powder, graphite, talcum, further silicone coatings different from the silicone material of the tube, silicone oil, graphite powder and/or Teflon powder dispersed in lubricating oil, as well as (e)PTFE and/or glass beads dispersed in lubricating oil, and a combination of any of these.

15. The method according to claim 14, further comprising the step of:
setting the surface of the treated tube, subsequent to the finish-treatment step but before the lubricating step, with at least one of: (e)PTFE beads and glass beads.

16. The method according to claim 1, wherein:
the tube is a shaft cover for an endoscope or an everting tube for an endoscope.

17. An endoscope having an everting tube drive, wherein:
the endoscope and/or the everting tube are/is made of a rubber material, characterized in that knob-shaped projections are formed on at least one of the surfaces of the everting tube and/or the surface of the endoscope.

18. The endoscope according to claim 17, wherein:
the rubber material is selected from silicone material, PTFE material, ePTFE material and thermoplastics, preferably thermoplastic polyurethane, into which preferably an oil is worked.

19. The endoscope according to claim 17, wherein:
a wax-containing fat or an oil is between and/or in the knob-shaped projections.

20. The endoscope according to claim 19, wherein:
the knob-shaped projections are formed as perforated lubricant reservoirs.

21. The endoscope according to claim 17, wherein:
the knob-shaped projections are formed by beads made of (e)PTFE or glass, which adhere to the respective surface.

22. The endoscope according to claim 21, wherein:
the beads have a bead diameter ranging from ≧0.1 to ≦0.2 mm.
